(19) United States
(12) Patent Application Publication
(10) Pub. No.: US 2024/0277302 A1
(43) Pub. Date: Aug. 22, 2024

(54) SENSOR DATA DISPLAY BOUNDARY DETERMINATION

(71) Applicant: Cardiac Pacemakers, Inc., St. Paul, MN (US)

(72) Inventors: Viktoria A. Averina, Shoreview, MN (US); Natalie Nicole Yee, Denver, CO (US); Kevin Chu, Shoreview, MN (US); Julie Lindmark, Shoreview, MN (US)

(21) Appl. No.: 18/441,857

(22) Filed: Feb. 14, 2024

Related U.S. Application Data
(60) Provisional application No. 63/446,972, filed on Feb. 20, 2023.

(51) Int. Cl. A61B 5/00; A61B 5/0205; A61B 5/08; A61N 1/37

(52) U.S. Cl. CPC A61B 5/742; A61B 5/0031; A61B 5/0205; A61B 5/686; A61B 5/7275; A61B 5/08; A61N 1/3702

(57) ABSTRACT

Systems and methods are disclosed to adjust one or more determined boundaries of a display of received physiologic information of a patient based on different first and second ranges of physiologic information, including determining first and second boundaries of a first range of the received physiologic information and first and second boundaries of a second range of the received physiologic information, wherein the second range is within the first range. The one or more determined boundaries of the display can be provided for presentation to a user, including the determined first and second boundaries of the first range and the determined first and second boundaries of the second range.

### CLAIM OF PRIORITY

This application claims the benefit of U.S. Provisional Application No. 63/446,972 filed on Feb. 20, 2023, which is hereby incorporated by reference in its entirety.

### TECHNICAL FIELD

This document relates generally to medical devices and more particularly to boundary determination for sensor data display.

### BACKGROUND

Ambulatory medical devices (AMDs), including implantable, subcutaneous, wearable, or one or more other medical devices, etc., can monitor, detect, or treat various conditions, including heart failure (HF), atrial fibrillation (AF), etc. Ambulatory medical devices can include sensors to sense physiological information from a patient and one or more circuits to detect one or more physiologic events using the sensed physiological information or transmit sensed physiologic information or detected physiologic events to one or more remote devices. Frequent patient monitoring can provide early detection of worsening patient condition, including worsening heart failure or atrial fibrillation.

Accurate identification of patients or groups of patients at an elevated risk of future adverse events may control mode or feature selection or resource management of one or more ambulatory medical devices, control notifications or messages in connected systems to various users associated with a specific patient or group of patients, organize or schedule physician or patient contact or treatment, or prevent or reduce patient hospitalization. Correctly identifying and safely managing patient risk of worsening condition may avoid unnecessary medical interventions, extend the usable life of ambulatory medical devices, and reduce healthcare costs. Further, improving the display of sensed physiologic information can reduce false determinations of patient condition or changes in patient condition, such as worsening patient condition, etc.

### SUMMARY

Systems and methods are disclosed to adjust one or more determined boundaries of a display of received physiologic information of a patient based on different first and second ranges of physiologic information, including determining first and second boundaries of a first range of the received physiologic information and first and second boundaries of a second range of the received physiologic information, wherein the second range is within the first range. The one or more determined boundaries of the display can be provided for presentation to a user, including the determined first and second boundaries of the first range and the determined first and second boundaries of the second range, such as for review of the received physiologic information.

An example of subject matter (e.g., a medical device system) may comprise a signal receiver circuit configured to receive physiologic information of a patient and an assessment circuit configured to adjust one or more determined boundaries of a display of the received physiologic information based on different first and second ranges of physiologic information, including to determine first and second boundaries of a first range of the received physiologic information and determine first and second boundaries of a second range of the received physiologic information, the second range within the first range.

In an example, the subject matter comprises an output circuit configured to provide the one or more determined boundaries of the display for presentation to a user, the display comprising the determined first and second boundaries of the first range and the determined first and second boundaries of the second range.

In an example, which may be combined with any one or more of the previous examples, the first boundary of the first range comprises a target value of the received physiologic information and the assessment circuit is configured to determine the target value as a function of a clinical target value and the received physiologic information.

In an example, which may be combined with any one or more of the previous examples, the assessment circuit is configured to update the one or more boundaries at a regular interval using additionally received physiologic information, wherein to update the one or more boundaries comprises to update the target value in a direction towards the clinical target, wherein the assessment circuit is restricted from updating the target value in a direction away from the clinical target.

In an example, which may be combined with any one or more of the previous examples, the assessment circuit is configured to determine the target value as a function of the clinical target value and a first extremum value of the received physiologic information occurring over the first range.

In an example, which may be combined with any one or more of the previous examples, the second boundary of the first range comprises a second extremum value of the received physiologic information occurring over the first range, wherein one of the first and second extremum value is a maximum value and another is a minimum value and the first and second boundaries of the second range comprise respective minimum and maximum values of the received physiologic information occurring over the second range.

In an example, which may be combined with any one or more of the previous examples, the output is configured to provide the first boundary as an arrow indicating a desired direction and the target value of the received physiologic information in contrast to the second range of the received physiologic information.

In an example, which may be combined with any one or more of the previous examples, the first and second boundaries of the first range comprise respective minimum and maximum values of the received physiologic information occurring over the first range and the first and second boundaries of the second range comprise respective minimum and maximum values of the received physiologic information occurring over the second range.

In an example, which may be combined with any one or more of the previous examples, the display comprises the determined first and second boundaries of the first range, the determined first and second boundaries of the second range, and a single representative current value of the received physiologic information.

In an example, which may be combined with any one or more of the previous examples, the single representative current value of the received physiologic information comprises a most recent daily value of the received physiologic information.

In an example, which may be combined with any one or more of the previous examples, the first range comprises a first number of daily values, the second range comprises a second number of daily values, the second number of daily values being more than two days and less than three weeks, and the first number of daily values is greater than the second number of daily values.

In an example, which may be combined with any one or more of the previous examples, the received physiologic information comprises daily values of the received physiologic information, the single representative current value of the received physiologic information comprises a most recent daily value of the received physiologic information, the first range comprises a previous six months of daily values with respect to the most recent daily value, and the second range comprises a previous two weeks of daily values with respect to the most recent daily value.

In an example, which may be combined with any one or more of the previous examples, the physiologic information includes at least one of respiration information of the patient, cardiac electrical information of the patient, impedance information of the patient, cardiac acceleration information of the patient, or sleep incline information of the patient.

In an example, which may be combined with any one or more of the previous examples, the physiologic information includes a plurality of daily respiration values and the display comprises the determined first and second boundaries of the first range, the determined first and second boundaries of the second range, and a most recent daily respiration value.

In an example, which may be combined with any one or more of the previous examples, a method includes receiving, using a signal receiver circuit, physiologic information of a patient, adjusting, using an assessment circuit, one or more determined boundaries of a display of the received physiologic information based on different first and second ranges of physiologic information, the adjusting comprising determining first and second boundaries of a first range of the received physiologic information and determining first and second boundaries of a second range of the received physiologic information, the second range within the first range, and providing, using an output circuit, the one or more determined boundaries of the display for presentation to a user, the display comprising the determined first and second boundaries of the first range and the determined first and second boundaries of the second range.

In an example, which may be combined with any one or more of the previous examples, determining the first boundary of the first range comprises determining a target value of the received physiologic information as a function of a clinical target value and the received physiologic information.

In an example, which may be combined with any one or more of the previous examples, the method includes updating, using the assessment circuit, the one or more boundaries at a regular interval using additionally received physiologic information, including updating the target value in a direction towards the clinical target and restricting updating the target value in a direction away from the clinical target.

In an example, which may be combined with any one or more of the previous examples, determining the target value includes as a function of the clinical target value and a first extremum value of the received physiologic information occurring over the first range.

In an example, which may be combined with any one or more of the previous examples, determining the second boundary of the first range comprises determining a second extremum value of the received physiologic information occurring over the first range, wherein one of the first and second extremum value is a maximum value and another is a minimum value and determining the first and second boundaries of the second range comprise determining respective minimum and maximum values of the received physiologic information occurring over the second range.

In an example, which may be combined with any one or more of the previous examples, providing the display comprises providing the first boundary as an arrow indicating a desired direction, and the target value, of the received physiologic information, in contrast to the second range of the received physiologic information.

In an example, which may be combined with any one or more of the previous examples, determining the first and second boundaries of the first range comprise determining respective minimum and maximum values of the received physiologic information occurring over the first range and determining the first and second boundaries of the second range comprise determining respective minimum and maximum values of the received physiologic information occurring over the second range.

In an example, which may be combined with any one or more of the previous examples, the display comprises the determined first and second boundaries of the first range, the determined first and second boundaries of the second range, and a single representative current value of the received physiologic information.

In an example, which may be combined with any one or more of the previous examples, the single representative current value of the received physiologic information comprises a most recent daily value of the received physiologic information.

In an example, which may be combined with any one or more of the previous examples, the second range comprises a first number of daily values, the first number of daily values being more than two days and less than three weeks and the first range comprises a second number of daily values, the second number greater than the first number of daily values.

In an example, which may be combined with any one or more of the previous examples, receiving the physiologic information comprises receiving daily values of the physiologic information, the single representative current value of the received physiologic information comprises a most recent daily value of the received physiologic information, the first range comprises a previous six months of daily values with respect to the most recent daily value, and the second range comprises a previous two weeks of daily values with respect to the most recent daily value.

In an example, which may be combined with any one or more of the previous examples, receiving the physiologic information includes receiving at least one of respiration information of the patient, cardiac electrical information of the patient, impedance information of the patient, cardiac acceleration information of the patient, or sleep incline information of the patient.

In an example, which may be combined with any one or more of the previous examples, receiving the physiologic information includes receiving a plurality of daily respiration values and providing the display comprises the providing the determined first and second boundaries of the first range, the determined first and second boundaries of the second range, and a most recent daily respiration value.

In an example, a system or apparatus may optionally combine any portion or combination of any portion of any one or more of the examples above to comprise “means for” performing any portion of any one or more of the functions or methods of the examples above, or at least one “non-transitory machine-readable medium” including instructions that, when performed by a machine, cause the machine to perform any portion of any one or more of the functions or methods of the examples above.

This summary is intended to provide an overview of subject matter of the present patent application. It is not intended to provide an exclusive or exhaustive explanation of the disclosure. The detailed description is included to provide further information about the present patent application. Other aspects of the disclosure will be apparent to persons skilled in the art upon reading and understanding the following detailed description and viewing the drawings that form a part thereof, each of which are not to be taken in a limiting sense.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates an example output to display physiologic information to a user.

FIGS. 2-4 illustrate example displays including one or more determined boundaries for different ranges of physiologic information.

FIG. 5 illustrates an example method for determining one or more boundaries of received physiologic information and providing a display of the determined boundaries to a user.

FIG. 6 illustrates an example implantable medical device (IMD) electrically coupled to a heart.

FIG. 7 illustrates an example medical device system.

FIG. 8 illustrates an example patient management system.

FIG. 9 illustrates a block diagram of an example machine upon which any one or more of the techniques discussed herein may perform.

### DETAILED DESCRIPTION

Ambulatory medical devices can include, or be configured to receive physiologic information from, one or more sensors located within, on, or proximate to a body of a patient. Physiologic information of the patient can include, among other things, respiration information (e.g., a respiratory rate, a respiration volume (tidal volume), cardiac acceleration information (e.g., cardiac vibration information, pressure waveform information, heart sound information, endocardial acceleration information, acceleration information, activity information, posture information, etc.); impedance information; cardiac electrical information; physical activity information (e.g., activity, steps, etc.); posture or position information; pressure information; plethysmograph information; chemical information; temperature information; or other physiologic information of the patient.

Sensed physiologic information is often detected, processed, stored, and transmitted to one or more displays for review by a user, such as a clinician or caregiver. However, display of received physiologic information, without reference to patient history or population benchmarks or thresholds, often provides an incomplete picture or understanding of patient condition. The opposite is also true, that gross comparison of patient physiologic information to optimal population thresholds may not show patient progress or changes in patient condition. Additionally, as displays for patient information are designed for a range of different devices with different screen sizes and available area, showing patient trends over long periods of time can reduce readability of the data. Even when a user is able to zoom in or adjust time scales or boundaries, such adjustment is time consuming, and having different user-adjustable scales and boundaries available may actually increase the time required for the user to understand patient progress or status, as the context of what is shown must be understood before inferences can be made.

The present inventors have recognized, among other things, systems and methods to display received physiologic information in a predictable, useful manner, such that patient condition can be easily, quickly, and repeatedly understood for a range of different data and patients, in a range of display area, from limited mobile displays to large monitors, such as for a user reviewing conditions of multiple patients, without user adjustment of scales or boundaries. For example, received physiologic information can be displayed based on two different ranges of received physiologic information, the second within the first. In addition, instead of merely using clinical target values for boundaries, the present inventors have recognized systems and methods to determine one or more boundaries of the different ranges using target values, where the target values are determined as a function of a clinical target value and the received physiologic information of the patient. In certain examples, patient progress towards the determined boundary can be displayed, including an indication of a desired direction (e.g., an arrow) for the physiologic information.

In certain examples, such as in monitoring different physiologic information associated with a specific disease or condition (e.g., heart failure, etc.), the ranges of different information may not scale with each other or move in unison (e.g., relative changes higher or lower can mean different things with the different physiologic information). In the example of heart failure, some key trends for display may include respiration information (e.g., respiratory rate), cardiac electrical information (e.g., heart rate, impedance, etc.), mechanical acceleration information (e.g., activity information, heart sounds, etc.), mechanical position information (e.g., patient posture, sleep incline, etc.).

In addition, the present inventors have recognized that frequently changing boundaries, or boundaries too far from the determined ranges, can be disadvantageous to users reviewing large amounts of information from one or more different patients, or in determining patient status at different times. Changing boundaries may provide misleading patient progress or status. Accordingly, the present inventors have recognized that, in certain examples, when updating the one or more determined boundaries including a target value (determined as a function of a clinical target value and the received physiologic information), such as using additional received physiologic information, that determined boundaries based on the target value can be updated if the new or updated target value approaches the clinical value, but not if the new or updated target value moves away from the clinical value. Restricting updates of determined boundaries away from the clinical value can reduce an incorrect appearance of patient improvement, not based on improving physiologic information, but on an updated worsening target value that makes. In certain examples, in response to a determined boundary moving away from a desired clinical target value, such as greater than a threshold amount, etc., indicating a determined worsening patient condition, an alert can be provided to the user or one or more additional processes or additional sensors can be triggered to improve patient monitoring. In other examples, sensing modes can be changed (e.g., from a low-power mode to a high-power mode, altering periods of detection of certain parameters, etc.), time periods of detected or determined parameters can be increased (e.g., detecting changes over longer time periods, such as multiple minutes, instead of seconds, etc.), sampling frequency or resolution of detected, sensed, or sampled information can be increased, data storage periods can be increased, or one or more notifications, alerts, therapies, or therapy parameters can be provided or changed based on the detected condition.

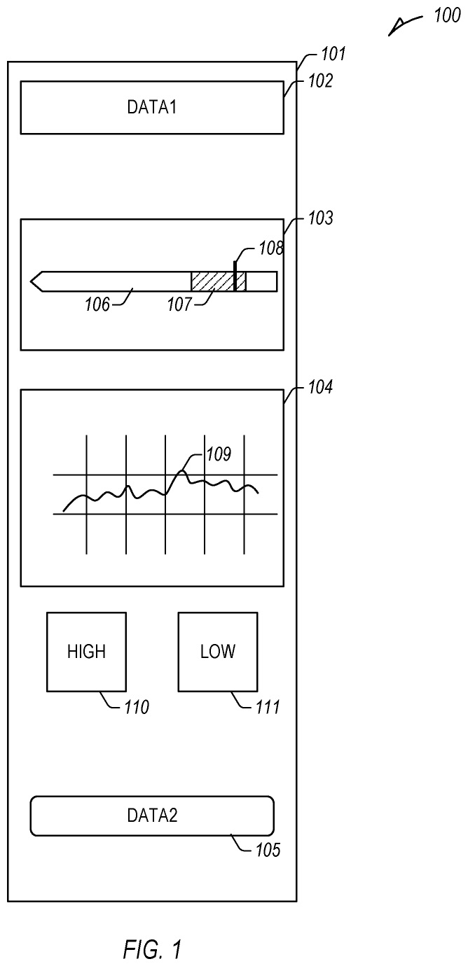

FIG. 1 illustrates an example output 100, such as a display of an application 101 on a user device (e.g., a mobile device, a medical device programmer, a medical device output, etc.), configured to display and communicate physiologic information of a patient (e.g., DATA1) in a first display 103 to a user, such as a clinician or other caregiver. A first field 102 can indicated the type of physiologic information of the patient displayed in the first display 103. In certain examples, the physiologic information of the patient can include one or more of respiratory rate, heart rate, activity, impedance, heart sound, patient posture or position or sleep incline information, etc. Although described herein with respect to physiologic information, and particularly DATA1 102, in certain examples, the display can include one or more parameters determined using patient physiologic information, such as one or more determined heart failure (HF) metrics, etc.

In an example, the first display 103 can include a major portion 106, such as a bar or other single-dimension display (e.g., a vertical bar, etc.) having first and second (outer) boundaries representative of received physiologic information of the patient over a first range, and a minor portion 107 having first and second (inner) boundaries representative of received physiologic information of the patient over a second range within the first range. The first and second ranges can include ranges of time. The minor portion 107 having the second range can be nested inside the major portion 106 having the first range, in certain examples, up to or including one or both of the first and second boundaries of the major portion 106, depending on the received physiologic information. In certain examples, the first display 103 can further include a representative value 108 of the received physiologic information, such as a single representative current value of the received physiologic information, a most recent daily value of the received physiologic information, etc.

The different ranges described herein with respect to the different major and minor portions 106, 107 of the physiologic information can include representations of physiologic information of the patient occurring over different time periods. For example, the first range of the major portion 106 can be a longer time period than the second range of the minor portion 107. In certain examples, the second range can be nested within the first range. However, in other examples, the second range can be non-overlapping with the first range but occurring over a time period more recent than the first range. For example, the first range can include a long-term range and the second range can include a short-term range, inside of or subsequent to the long-term range. In contrast, the representative value 108 can include a value representative of a third range shorter than the second range, such as a single measurement, a most recent measurement, a most recent daily value, etc.

In certain examples, the output 100 can include a second display 104 illustrating a trend 109 of the received physiologic information over time, respective high and low patient values 110, 111, or one or more other displays, etc. The output 100 can optionally include a selection 105 to transition from displaying DATA1 to DATA2.

FIGS. 2-4 illustrate different examples of portions of an output 200, 300, 400 including one or more determined boundaries for different ranges of physiologic information. Although different physiologic information includes different natural variation with different scales and magnitudes, and the values of such information vary differently for each patient, the boundaries for each of the major and minor portions 106, 107 can be determined to quickly and easily display an indication of patient condition or status (e.g., worsening, improving, etc.) without reviewing historical trends or normalizing individual values of short-term or long-term ranges. Additionally, the width of each of the major and minor portions 106, 107, and the proportions between them, can themselves be indicative of variation in the individual ranges, an advantage over single representative values or even displayed trends.

FIG. 2 illustrates an example portion of an output 200, similar to the first display 103 of FIG. 1. In addition to the major and minor portions 106, 107 and the representative value 108 of the physiologic information described above in FIG. 1, the output 200 includes respective first and second boundaries 112, 113 of the major portion 106 and first and second boundaries 117, 118 of the minor portion 107. In an example, a value of one more of boundaries can be illustrated, such as the first and second boundary values 114, 115 of the major portion 106, etc.

In the most basic example, the first and second boundaries of the major and minor portions 106, 107 can include the maximum and minimum values of the physiologic information occurring over the first and second ranges. However, in certain examples using the maximum and minimum alone does not communicate a comparison to a clinical target value. In other examples, setting at least one of the boundaries of the major or minor portions 106, 107 to one or more clinical target values may provide inaccurate presentation of patient physiologic information and patient condition.

For example, for the purpose of discussion, the physiologic information displayed in FIG. 2 can include respiration information, such as respiratory rate (breaths per minute). Although described herein with respect to such, in other examples, one or more other types of physiologic information can be displayed. Typically, different physiologic information have different clinical target values or ranges. For example, for respiratory rate, a clinical target value can be 12 breaths per minute. However, certain patients experience respiratory rates a substantial distance away from the clinical target value (e.g., 20-24 breaths per minute, etc.). In those examples, where the clinical target value is a substantial distance from the range of values experienced by the patient over one or both of the first or second ranges, if the clinical target value is set as the first boundary 112 of the major portion 106, the minor portion 107 can appear in the display as being narrower (a tighter group) than it actually is. Further, the range of values experienced by the patient over the first range in the major portion 106 may not be an accurate representation of that experienced by the patient.

Accordingly, the present inventors have recognized that one or more target values can be determined for at least one boundary of the major portion 106 as a function of (1) a clinical target value received for the respective physiologic information of the patient and (2) the received physiologic information of the patient, such as occurring over the first range of the major portion 106. In the example of FIG. 2, the first boundary 112 can include the target value, indicated with an arrow indicating the desired direction of the target value, and the second boundary 113 can include a maximum value of the physiologic information occurring over the first range.

In an example, the target value can be described by the following function:

$$target value = min ( Z , max ( Y - A , C ) ) \qquad ( 1 )$$

where Y is the patient's minimum value of the physiologic information over the first range, C is the clinical target value of the physiologic information, A is a threshold amount from the clinical target value (in certain examples determined as a function of C), and Z is a patient minimum value of the physiologic information.

For example, with respect to respiratory rate, the clinical target value can be 12 breaths per minute (bpm) and the threshold amount can be two bpm. If the minimum value of the respiratory rate of the patient over the first range is 18 bpm, and the patient minimum value of the physiologic information is greater than 16 bpm, then the target value can be computed as:

$$target value = min ( Z , max ( 18 - 2 , 12 ) ) = 16 \qquad ( 2 )$$

FIG. 2 illustrates the first boundary 112 of the major portion 106 as the determined target value of 16 bpm, as additionally illustrated by the first boundary value 114, and the second boundary 113 of the major portion 106 as a maximum value of the respiratory rate occurring over the first range of 22 bpm, as additionally illustrated by the second boundary value 115. FIG. 2 additionally illustrates the first boundary 117 of the minor portion 107 as approximately 19 bpm, the second boundary 118 of the minor portion 107 as approximately 21 bpm, and the representative value 108 (e.g., a most recent daily value) as greater than 20 but less than 21 bpm. The output 200 in FIG. 2 indicates a generally poor (but not worst case) patient condition, not at the maximum value of the first range, but generally closer to each of the second boundaries 113, 118 of the major and minor portions 106, 107 than the first boundaries 112, 117 of the major and minor portions 106, 107.

In an example, the minor portion 107 can illustrate patient physiologic information over the last 14 days (e.g., a short-term range), the major portion 106 can illustrate patient physiologic information over the last 6 months (e.g., a long-term range), including the first boundary as a target value, even if the target value was never reached. In certain examples, the target value can be initially determined by the above function at initialization (e.g., over two weeks after implant). Each boundary can be updated regularly (e.g., daily, weekly, using the received information over the first and second ranges). In certain examples, the target value can be updates with an option to only update in the direction towards the clinical target value, and not away from the clinical target value.

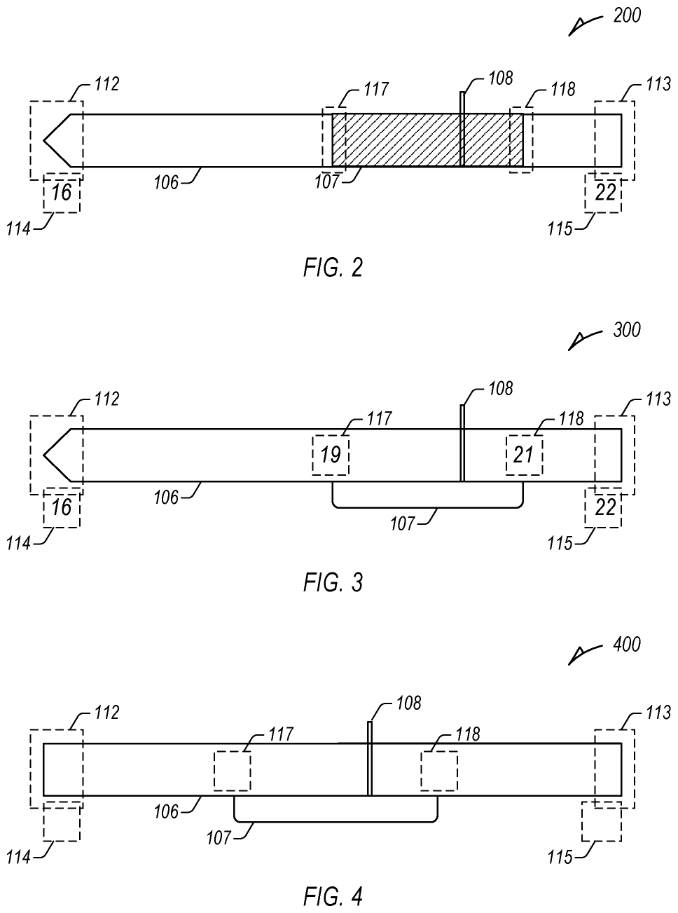

FIG. 3 illustrates an example portion of an output 300, similar to the output 200 in FIG. 2, only with the minor portion 107 indicated by a different marker. In other examples, one or more other graphical features or markers can be used to distinguish the major and minor portions 106, 107.

FIG. 4 illustrates an example portion of an output 400, illustrating different physiologic information than that illustrated in FIGS. 1-3, for example heart rate of the patient having a clinical target value or range inside that major or minor portions 106, 107, and thus without arrows at the outer boundaries, in contrast to that with respect to the respiratory rate of the patient illustrated in FIGS. 1-3.

The first and second boundaries 112, 113 of the major portion 106 have first and second boundary values 114, 115 of 30 and 120 beats per minute (bpm) respectively, the first and second boundaries 117, 118 of the minor portion 107 are 60 and 90 bpm, and the representative value 108 (e.g., a most recent daily value) as approximately 80 bpm. In an example, the first and second boundaries 112, 113 can be minimum and maximum values of the heart rate of the patient occurring over the first range. In other examples, the first and second boundaries 112, 113 can each be target values (e.g., indicated using arrows) computed using a function similar to that described above with respect to the respiratory rate, only reflective of both low and high clinical targets of the output 400 illustrated in FIG. 4, instead of only the lower clinical target illustrated in FIG. 2.

In certain examples, the different outputs illustrated in the examples of FIGS. 1-4 can include major or minor portions having different shading to distinguish the different portions. However, in certain examples, the respective portions themselves can include a gradient or shading according to the values represented or illustrated thereby, with colors or shading changing according to a normality or abnormality of the value in contrast to a clinical target value or range. Although illustrated and discussed herein with respect to daily values, in certain examples such values can include frequencies, timings, or other measures.

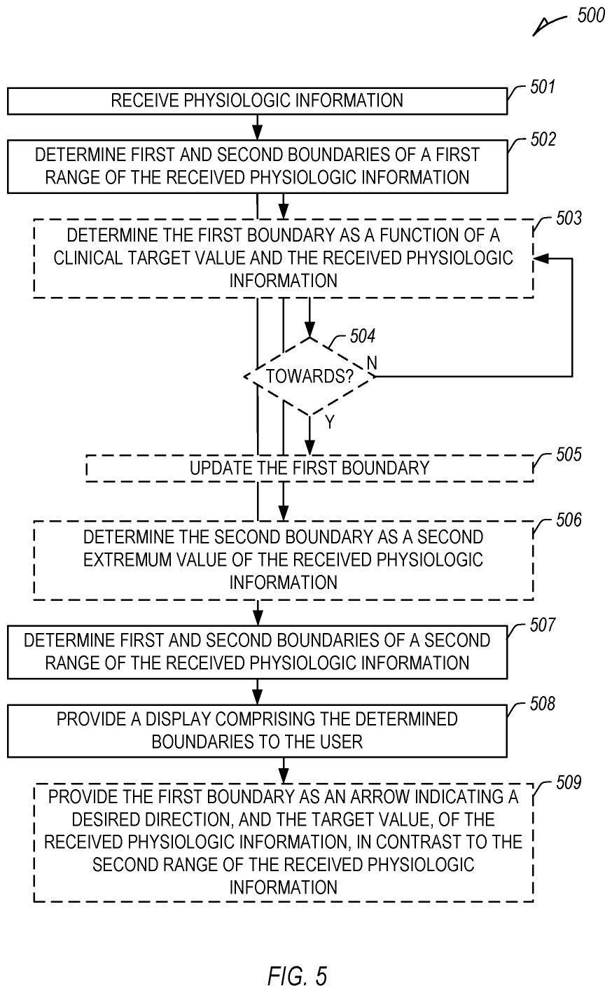

FIG. 5 illustrates an example method 500 for determining or adjusting one or more boundaries of a display of received physiologic information based on different first and second ranges of the received physiologic information, such as using one or more of a signal receiver circuit or an assessment circuit, and providing a display of the determined boundaries to a user, such as using an output comprising a display screen, etc.

At step 501, physiologic information of a patient can be received, such as using a signal receiver circuit. The physiologic information of the patient can include at least one of respiration information (e.g., respiratory rate, tidal volume, RSBI, etc.), cardiac electrical information (e.g., heart rate, impedance, etc.), impedance information, cardiac acceleration information (e.g., heart sounds, etc.), mechanical acceleration information (e.g., activity information, heart sounds, etc.), mechanical position information (e.g., patient posture, sleep incline, etc.).

At step 502, first and second boundaries of a first range of the received physiologic information can be determined. The first range can include a long-term time period greater than a short-term time period. In certain examples, the long-term time period can include a time period comprising a number of days greater than a week, and in certain examples, greater than two weeks, greater than three weeks, greater than a month, greater than two months, etc. In one example, the long-term time period includes a previous six months of daily values with respect to a most recent daily value. In another example, the long-term time period can include an entire patient history of received physiologic information.

At step 503, one or more of the boundaries, such as the first boundary of the first range, can be optionally determined as a target value of the received physiologic information. The target value can be determined as a function of a clinical target value and the received physiologic information. In certain examples, the clinical target value can be received as a predetermined clinical target value, such as a clinical target value determined based on information from one or more medical device systems comparing physiologic information from patients grouped as having similar diagnoses, demographic values or other information about the patient or patient condition, etc. or otherwise received from or set by a clinician. In an example, the target value can be determined, such as by the assessment circuit, etc., as a function of the clinical target value and a first extremum value of the received physiologic information occurring over the first range. The first extremum value can be a maximum value or a minimum value depending on a relationship between the physiologic information of the patient and the clinical target value.

In certain examples, the target value can be re-determined at a regular interval, such as daily, weekly, or at one or more intervals, etc. using additionally received physiologic information of the patient. In certain examples, the target value can be updated in a direction towards the clinical target but can be restricted from being updated in a direction away from the clinical target.

At step 504, if the re-determined target value moves away from the clinical value (in contrast to the previously determined target value), then process can optionally return to step 503. For example, if the clinical value is 12 breaths per minute, the previous target value (N−1) is 16 breaths per minute, and the new target value (N) is 17 breaths per minute, then the re-determined target value has moved away from the clinical value, and not towards the target value. However, if the new target value (N) is 15 breaths per minute, then the re-determined target value has moved towards the clinical value, and the process can optionally proceed to step 505. In certain examples, if the target value is below the clinical value, the lower of the two values can be used. In other examples, the target value can include a minimum value of the physiologic information of the patient over the first range, or in other examples, over all received physiologic information of the patient, even outside of the first range.

At step 505, if the re-determined target value moves towards the clinical value (in contrast to the previously determined target value), then the first boundary can be optionally updated to the re-determined target value. In certain examples, one or more other boundaries can be updated at a regular interval using additionally received physiologic information of the patient, regardless of a direction of movement.

At step 506, the second boundary of the first range can be optionally determined as a second extremum value of the received physiologic information occurring over the first range. The second extremum value can be a maximum value or a minimum value, generally opposing the first extremum value.

At step 507, first and second boundaries of a second range of the received physiologic information can be determined. The second range can include a short-term period shorter than the first range. In certain examples, the short-term period can include a time period comprising a number of days greater than two days but less than three weeks, such as two weeks, etc. In one example, the short-term time period includes a previous two weeks of daily values with respect to a most recent daily value. In an example, the second range can include a most recent short-term time period within the first range. In other examples, the first and second ranges can be non-overlapping with the first range preceding the second range. In an example, the first and second boundaries of the second range can be determined as respective minimum and maximum values of the received physiologic information occurring over the second range.

At step 508, the one or more determined boundaries of the display can be provided for presentation to a user, such as using an output circuit, the display comprising the determined first and second boundaries of the first range and the determined first and second boundaries of the second range.

In an example, the display can include the determined first and second boundaries of the first range, the determined first and second boundaries of the second range, and a single representative current value of the received physiologic information. In certain examples, the received physiologic information can include daily values of the received physiologic information and the single representative current value of the received physiologic information can include a most recent daily value of the received physiologic information.

At step 509, the first boundary can be optionally provided as an arrow indicating a desired direction of the displayed information (e.g., towards the clinical target value) and the target value of the received physiologic information in contrast to the second range of the received physiologic information.

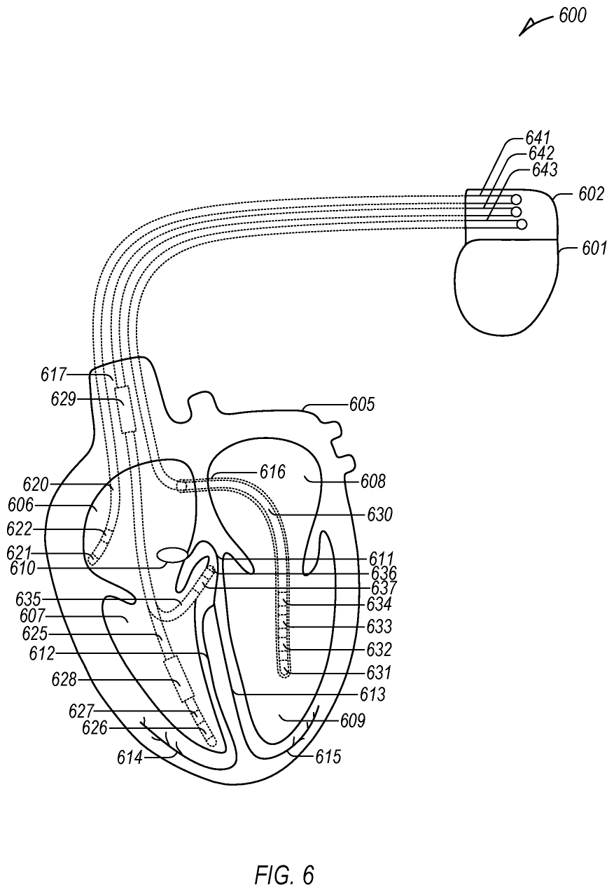

FIG. 6 illustrates an implantable medical device (IMD) 600 electrically coupled to a heart 605, such as through one or more leads coupled to the IMD 600 through one or more lead ports, such as first, second, or third lead ports 641, 642, 643 in a header 602 of the IMD 600. In an example, the IMD 600 can include an antenna, such as in the header 602, configured to enable communication with an external system and one or more electronic circuits (e.g., an assessment circuit, etc.) in a hermetically sealed housing (CAN) 601. The IMD 600 illustrates an example medical device (or a medical device system) as described herein.

The IMD 600 may include an implantable medical device (IMD), such as an implantable cardiac monitor (ICM), pacemaker, defibrillator, cardiac resynchronizer, or other subcutaneous IMD or cardiac rhythm management (CRM) device configured to be implanted in a chest of a subject, having one or more leads to position one or more electrodes or other sensors at various locations in or near the heart 605, such as in one or more of the atria or ventricles. Separate from, or in addition to, the one or more electrodes or other sensors of the leads, the IMD 600 can include one or more electrodes or other sensors (e.g., a pressure sensor, an accelerometer, a gyroscope, a microphone, etc.) powered by a power source in the IMD 600. The one or more electrodes or other sensors of the leads, the IMD 600, or a combination thereof, can be configured detect physiologic information from, or provide one or more therapies or stimulation to, the patient.

The IMD 600 can include one or more electronic circuits configured to sense one or more physiologic signals, such as an electrogram or a signal representing mechanical function of the heart 605. In certain examples, the CAN 601 may function as an electrode such as for sensing or pulse delivery. For example, an electrode from one or more of the leads may be used together with the CAN 601 such as for unipolar sensing of an electrogram or for delivering one or more pacing pulses. A defibrillation electrode (e.g., the first defibrillation coil electrode 628, the second defibrillation coil electrode 629, etc.) may be used together with the CAN 601 to deliver one or more cardioversion/defibrillation pulses.

In an example, the IMD 600 can sense impedance such as between electrodes located on one or more of the leads or the CAN 601. The IMD 600 can be configured to inject current between a pair of electrodes, sense the resultant voltage between the same or different pair of electrodes, and determine impedance, such as using Ohm's Law. The impedance can be sensed in a bipolar configuration in which the same pair of electrodes can be used for injecting current and sensing voltage, a tripolar configuration in which the pair of electrodes for current injection and the pair of electrodes for voltage sensing can share a common electrode, or tetrapolar configuration in which the electrodes used for current injection can be distinct from the electrodes used for voltage sensing, etc. In an example, the IMD 600 can be configured to inject current between an electrode on one or more of the first, second, third, or fourth leads 620, 625, 630, 635 and the CAN 601, and to sense the resultant voltage between the same or different electrodes and the CAN 601.

The example lead configurations in FIG. 6 include first, second, and third leads 620, 625, 630 in traditional lead placements in the right atrium (RA) 606, right ventricle (RV) 607, and in a coronary vein 616 (e.g., the coronary sinus) over the left atrium (LA) 608 and left ventricle (LV) 609, respectively, and a fourth lead 635 positioned in the RV 607 near the His bundle 611, between the AV node 610 and the right and left bundle branches 612, 613 and Purkinje fibers 614, 615. Each lead can be configured to position one or more electrodes or other sensors at various locations in or near the heart 605 to detect physiologic information or provide one or more therapies or stimulation.

The first lead 620, positioned in the RA 606, includes a first tip electrode 621 located at or near the distal end of the first lead 620 and a first ring electrode 622 located near the first tip electrode 621. The second lead 625 (dashed), positioned in the RV 607, includes a second tip electrode 626 located at or near the distal end of the second lead 625 and a second ring electrode 627 located near the second tip electrode 626. The third lead 630, positioned in the coronary vein 616 over the LV 609, includes a third tip electrode 631 located at or near the distal end of the third lead 630, a third ring electrode 632 located near the third tip electrode 631, and two additional electrodes 633, 634. The fourth lead 635, positioned in the RV 607 near the His bundle 611, includes a fourth tip electrode 636 located at or near the distal end of the fourth lead 635 and a fourth ring electrode 637 located near the fourth tip electrode 636. The tip and ring electrodes can include pacing/sensing electrodes configured to sense electrical activity or provide pacing stimulation.

In addition to tip and ring electrodes, one or more leads can include one or more defibrillation coil electrodes configured to sense electrical activity or provide cardioversion or defibrillation shock energy. For example, the second lead 625 includes a first defibrillation coil electrode 628 located near the distal end of the second lead 625 in the RV 607 and a second defibrillation coil electrode 629 located a distance from the distal end of the second lead 625, such as for placement in or near the superior vena cava (SVC) 617.

Different CRM devices include different number of leads and lead placements. For examples, some CRM devices are single-lead devices having one lead (e.g., RV only, RA only, etc.). Other CRM devices are multiple-lead devices having two or more leads (e.g., RA and RV; RV and LV; RA, RV, and LV; etc.). CRM devices adapted for His bundle pacing often use lead ports designated for LV or RV leads to deliver stimulation to the His bundle 611.

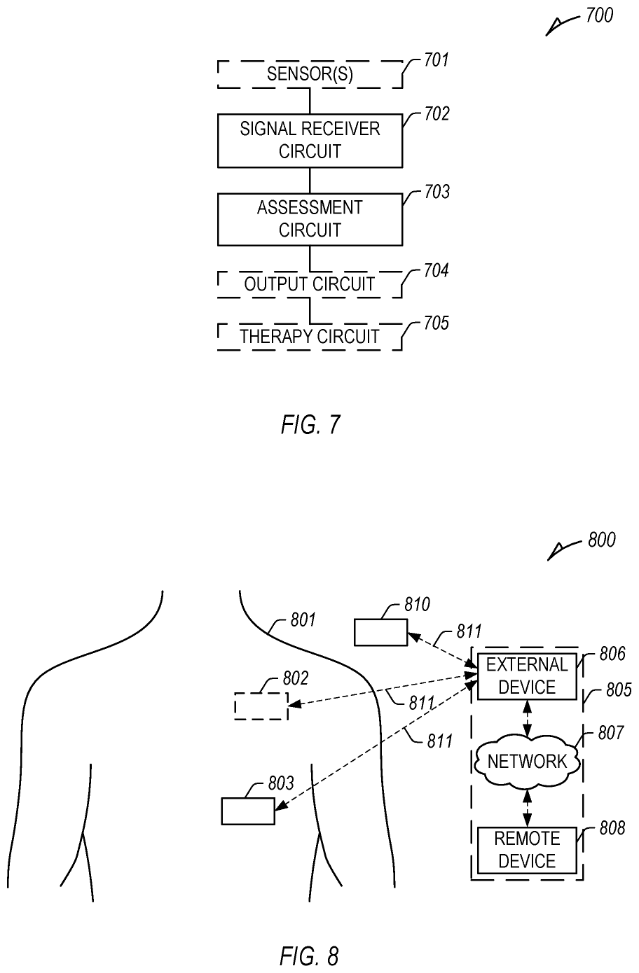

FIG. 7 illustrates an example system 700 (e.g., a medical device system). In an example, one or more aspects of the example system 700 can be a component of, or communicatively coupled to, a medical device, such as an implantable medical device (IMD), an insertable cardiac monitor, an ambulatory medical device (AMD), etc. The system 700 can be configured to monitor, detect, or treat various physiologic conditions of the body, such as cardiac conditions associated with a reduced ability of a heart to sufficiently deliver blood to a body, including heart failure, arrhythmias, dyssynchrony, etc., or one or more other physiologic conditions and, in certain examples, can be configured to provide electrical stimulation or one or more other therapies or treatments to the patient.

The system 700 can include a single medical device or a plurality of medical devices implanted in a patient's body or otherwise positioned on or about the patient to monitor patient physiologic information of the patient using information from one or more sensors, such as a sensor 701. In an example, the sensor 701 can include one or more of: a respiration sensor configured to receive respiration information (e.g., a respiratory rate, a respiration volume (tidal volume), etc.); an acceleration sensor (e.g., an accelerometer, a microphone, etc.) configured to receive cardiac acceleration information (e.g., cardiac vibration information, pressure waveform information, heart sound information, endocardial acceleration information, acceleration information, activity information, posture information, etc.); an impedance sensor (e.g., an intrathoracic impedance sensor, a transthoracic impedance sensor, a thoracic impedance sensor, etc.) configured to receive impedance information; a cardiac sensor configured to receive cardiac electrical information; an activity sensor configured to receive information about a physical motion (e.g., activity, steps, etc.); a posture sensor configured to receive posture or position information; a pressure sensor configured to receive pressure information; a plethysmograph sensor (e.g., a photoplethysmography sensor, etc.); a chemical sensor (e.g., an electrolyte sensor, a pH sensor, an anion gap sensor, etc.); a temperature sensor; a skin elasticity sensor, or one or more other sensors configured to receive physiologic information of the patient.

The example system 700 can include a signal receiver circuit 702 and an assessment circuit 703. The signal receiver circuit 702 can be configured to receive physiologic information of a patient (or group of patients) from the sensor 701. The assessment circuit 703 can be configured to receive information from the signal receiver circuit 702, and to determine one or more parameters (e.g., physiologic parameters, stratifiers, etc.) or existing or changed patient conditions (e.g., indications of patient dehydration, respiratory condition, cardiac condition (e.g., heart failure, arrhythmia), sleep disordered breathing, etc.) using the received physiologic information, such as described herein. The physiologic information can include, among other things, cardiac electrical information, impedance information, respiration information, heart sound information, activity information, posture information, temperature information, or one or more other types of physiologic information.

In certain examples, the assessment circuit 703 can aggregate information from multiple sensors or devices, detect various events using information from each sensor or device separately or in combination, update a detection status for one or more patients based on the information, and transmit a message or an alert to one or more remote devices that a detection for the one or more patients has been made or that information has been stored or transmitted, such that one or more additional processes or systems can use the stored or transmitted detection or information for one or more other review or processes.

In certain examples, such as to detect an improved or worsening patient condition, some initial assessment is often required to establish a baseline level or condition from one or more sensors or physiologic information. Subsequent detection of a deviation from the baseline level or condition can be used to determine the improved or worsening patient condition. However, in other examples, the amount of variation or change (e.g., relative or absolute change) in physiologic information over different time periods can used to determine a risk of an adverse medical event, or to predict or stratify the risk of the patient experiencing an adverse medical event (e.g., a heart failure event) in a period following the detected change, in combination with or separate from any baseline level or condition.

Changes in different physiologic information can be aggregated and weighted based on one or more patient-specific stratifiers and, in certain examples, compared to one or more thresholds, for example, having a clinical sensitivity and specificity across a target population with respect to a specific condition (e.g., heart failure), etc., and one or more specific time periods, such as daily values, short term averages (e.g., daily values aggregated over a number of days), long term averages (e.g., daily values aggregated over a number of short term periods or a greater number of days (sometimes different (e.g., non-overlapping) days than used for the short term average)), etc.

The system 700 can include an output circuit 704 configured to provide an output to a user, or to cause an output to be provided to a user, such as through an output, a display, or one or more other user interface, the output including a score, a trend, an alert, or other indication. In other examples, the output circuit 704 can be configured to provide an output to another circuit, machine, or process, such as a therapy circuit 705 (e.g., a cardiac resynchronization therapy (CRT) circuit, a chemical therapy circuit, a stimulation circuit, etc.), etc., to control, adjust, or cease a therapy of a medical device, a drug delivery system, etc., or otherwise alter one or more processes or functions of one or more other aspects of a medical device system, such as one or more CRT parameters, drug delivery, dosage determinations or recommendations, etc. In an example, the therapy circuit 705 can include one or more of a stimulation control circuit, a cardiac stimulation circuit, a neural stimulation circuit, a dosage determination or control circuit, etc. In other examples, the therapy circuit 705 can be controlled by the assessment circuit 703, or one or more other circuits, etc. In certain examples, the assessment circuit 703 can include the output circuit 704 or can be configured to determine the output to be provided by the output circuit 704, while the output circuit 704 can provide the signals that cause the user interface to provide the output to the user based on the output determined by the assessment circuit 703.

A technological problem exists in medical devices and medical device systems that in low-power monitoring modes, ambulatory medical devices powered by one or more rechargeable or non-rechargeable batteries (e.g., including IMDs) have to make certain tradeoffs between battery life, or in the instance of implantable medical devices with non-rechargeable batteries, between device replacement periods often including surgical procedures, and sampling resolution, sampling periods, of processing, storage, and transmission of sensed physiologic information, or features or mode selection of or within the medical devices. Medical devices can include higher-power modes and lower-power modes. Physiologic information, such as indicative of a potential adverse physiologic event, can be used to transition from a low-power mode to a high-power mode. In certain examples, the low-power mode can include a low resource mode, characterized as requiring less power, processing time, memory, or communication time or bandwidth (e.g., transferring less data, etc.) than a corresponding high-power mode. The high-power mode can include a relatively higher resource mode, characterized as requiring more power, processing time, memory, or communication time or bandwidth than the corresponding low-power mode. However, by the time physiologic information detected in the low-power mode indicates a possible event, valuable information has been lost, unable to be recorded in the high-power mode.

The inverse is also true, in that false or inaccurate determinations that trigger a high-power mode unnecessarily unduly limit the usable life of certain ambulatory medical devices. For numerous reasons, it is advantageous to accurately detect and determine physiologic events, and to avoid unnecessary transitions from the low-power mode to the high-power mode to improve use of medical device resources.

For example, a change in modes can enable higher resolution sampling or an increase in the sampling frequency or number or types of sensors used to sense physiologic information leading up to and including a potential event. For example, different physiologic information is often sensed using non-overlapping time periods of the same sensor, in certain examples, at different sampling frequencies and power costs. In one example, heart sounds and patient activity can be detected using non-overlapping time periods of the same, single- or multi-axis accelerometer, at different sampling frequencies and power costs. In certain examples, a transition to a high-power mode can include using the accelerometer to detect heart sounds throughout the high-power mode, or at a larger percentage of the high-power mode than during a corresponding low-power mode, etc. In other examples, waveforms for medical events can be recorded, stored in long-term memory, and transferred to a remote device for clinician review. In certain examples, only a notification that an event has been stored is transferred, or summary information about the event. In response, the full event can be requested for subsequent transmission and review. However, even in the situation where the event is stored and not transmitted, resources for storing and processing the event are still by the medical device.

FIG. 8 illustrates an example patient management system 800 and portions of an environment in which the patient management system 800 may operate. The patient management system 800 can perform a range of activities, including remote patient monitoring and diagnosis of a disease condition. Such activities can be performed proximal to a patient 801, such as in a patient home or office, through a centralized server, such as in a hospital, clinic, or physician office, or through a remote workstation, such as a secure wireless mobile computing device.

The patient management system 800 can include one or more medical devices, an external system 805, and a communication link 811 providing for communication between the one or more ambulatory medical devices and the external system 805. The one or more medical devices can include an ambulatory medical device (AMD), such as an implantable medical device (IMD) 802, a wearable medical device 803, or one or more other implantable, leadless, subcutaneous, external, wearable, or medical devices configured to monitor, sense, or detect information from, determine physiologic information about, or provide one or more therapies to treat various conditions of the patient 801, such as one or more cardiac or non-cardiac conditions (e.g., dehydration, sleep disordered breathing, etc.).

In an example, the implantable medical device 802 can include one or more cardiac rhythm management devices implanted in a chest of a patient, having a lead system including one or more transvenous, subcutaneous, or non-invasive leads or catheters to position one or more electrodes or other sensors (e.g., a heart sound sensor) in, on, or about a heart or one or more other position in a thorax, abdomen, or neck of the patient 801. In another example, the implantable medical device 802 can include a monitor implanted, for example, subcutaneously in the chest of patient 801, the implantable medical device 802 including a housing containing circuitry and, in certain examples, one or more sensors, such as a temperature sensor, etc.

Cardiac rhythm management devices, such as insertable cardiac monitors, pacemakers, defibrillators, or cardiac resynchronizers, include implantable or subcutaneous devices having hermetically sealed housings configured to be implanted in a chest of a patient. The cardiac rhythm management device can include one or more leads to position one or more electrodes or other sensors at various locations in or near the heart, such as in one or more of the atria or ventricles of a heart, etc. Accordingly, cardiac rhythm management devices can include aspects located subcutaneously, though proximate the distal skin of the patient, as well as aspects, such as leads or electrodes, located near one or more organs of the patient. Separate from, or in addition to, the one or more electrodes or other sensors of the leads, the cardiac rhythm management device can include one or more electrodes or other sensors (e.g., a pressure sensor, an accelerometer, a gyroscope, a microphone, etc.) powered by a power source in the cardiac rhythm management device. The one or more electrodes or other sensors of the leads, the cardiac rhythm management device, or a combination thereof, can be configured detect physiologic information from the patient, or provide one or more therapies or stimulation to the patient.

Implantable devices can additionally or separately include leadless cardiac pacemakers (LCPs), small (e.g., smaller than traditional implantable cardiac rhythm management devices, in certain examples having a volume of about 1 cc, etc.), self-contained devices including one or more sensors, circuits, or electrodes configured to monitor physiologic information (e.g., heart rate, etc.) from, detect physiologic conditions (e.g., tachycardia) associated with, or provide one or more therapies or stimulation to the heart without traditional lead or implantable cardiac rhythm management device complications (e.g., required incision and pocket, complications associated with lead placement, breakage, or migration, etc.). In certain examples, leadless cardiac pacemakers can have more limited power and processing capabilities than a traditional cardiac rhythm management device; however, multiple leadless cardiac pacemakers can be implanted in or about the heart to detect physiologic information from, or provide one or more therapies or stimulation to, one or more chambers of the heart. The multiple leadless cardiac pacemaker can communicate between themselves, or one or more other implanted or external devices.

The implantable medical device 802 can include an assessment circuit configured to detect or determine specific physiologic information of the patient 801, or to determine one or more conditions or provide information or an alert to a user, such as the patient 801 (e.g., a patient), a clinician, or one or more other caregivers or processes, such as described herein. The implantable medical device 802 can alternatively or additionally be configured as a therapeutic device configured to treat one or more medical conditions of the patient 801. The therapy can be delivered to the patient 801 via the lead system and associated electrodes or using one or more other delivery mechanisms. The therapy can include delivery of one or more drugs to the patient 801, such as using the implantable medical device 802 or one or more of the other ambulatory medical devices, etc. In some examples, therapy can include CRT for rectifying dyssynchrony and improving cardiac function in heart failure patients. In other examples, the implantable medical device 802 can include a drug delivery system, such as a drug infusion pump to deliver drugs to the patient for managing arrhythmias or complications from arrhythmias, hypertension, hypotension, or one or more other physiologic conditions. In other examples, the implantable medical device 802 can include one or more electrodes configured to stimulate the nervous system of the patient or to provide stimulation to the muscles of the patient airway, etc.

The wearable medical device 803 can include one or more wearable or external medical sensors or devices (e.g., automatic external defibrillators (AEDs), Holter monitors, patch-based devices, smart watches, smart accessories, wrist- or finger-worn medical devices, such as a finger-based photoplethysmography sensor, etc.).

The external system 805 can include a dedicated hardware/software system, such as a programmer, a remote server-based patient management system, or alternatively a system defined predominantly by software running on a standard personal computer. The external system 805 can manage the patient 801 through the implantable medical device 802 or one or more other ambulatory medical devices connected to the external system 805 via a communication link 811. In other examples, the implantable medical device 802 can be connected to the wearable medical device 803, or the wearable medical device 803 can be connected to the external system 805, via the communication link 811. This can include, for example, programming the implantable medical device 802 to perform one or more of acquiring physiologic data, performing at least one self-diagnostic test (such as for a device operational status), analyzing the physiologic data, or optionally delivering or adjusting a therapy for the patient 801. Additionally, the external system 805 can send information to, or receive information from, the implantable medical device 802 or the wearable medical device 803 via the communication link 811. Examples of the information can include real-time or stored physiologic data from the patient 801, diagnostic data, such as detection of patient hydration status, hospitalizations, responses to therapies delivered to the patient 801, or device operational status of the implantable medical device 802 or the wearable medical device 803 (e.g., battery status, lead impedance, etc.). The communication link 811 can be an inductive telemetry link, a capacitive telemetry link, or a radio-frequency (RF) telemetry link, or wireless telemetry based on, for example, “strong” Bluetooth or IEEE 602.11 wireless fidelity “Wi-Fi” interfacing standards. Other configurations and combinations of patient data source interfacing are possible.

The external system 805 can include an external device 806 in proximity of the one or more ambulatory medical devices, and a remote device 808 in a location relatively distant from the one or more ambulatory medical devices, in communication with the external device 806 via a communication network 807. Examples of the external device 806 can include a medical device programmer. The remote device 808 can be configured to evaluate collected patient or patient information and provide alert notifications, among other possible functions. In an example, the remote device 808 can include a centralized server acting as a central hub for collected data storage and analysis from a number of different sources. Combinations of information from the multiple sources can be used to make determinations and update individual patient status or to adjust one or more alerts or determinations for one or more other patients. The server can be configured as a uni-, multi-, or distributed computing and processing system. The remote device 808 can receive data from multiple patients. The data can be collected by the one or more ambulatory medical devices, among other data acquisition sensors or devices associated with the patient 801. The server can include a memory device to store the data in a patient database. The server can include an alert analyzer circuit to evaluate the collected data to determine if specific alert condition is satisfied. Satisfaction of the alert condition may trigger a generation of alert notifications, such to be provided by one or more human-perceptible user interfaces. In some examples, the alert conditions may alternatively or additionally be evaluated by the one or more ambulatory medical devices, such as the implantable medical device. By way of example, alert notifications can include a Web page update, phone or pager call, E-mail, SMS, text or “Instant” message, as well as a message to the patient and a simultaneous direct notification to emergency services and to the clinician. Other alert notifications are possible. The server can include an alert prioritizer circuit configured to prioritize the alert notifications. For example, an alert of a detected medical event can be prioritized using a similarity metric between the physiologic data associated with the detected medical event to physiologic data associated with the historical alerts.

The remote device 808 may additionally include one or more locally configured clients or remote clients securely connected over the communication network 807 to the server. Examples of the clients can include personal desktops, notebook computers, mobile devices, or other computing devices. System users, such as clinicians or other qualified medical specialists, may use the clients to securely access stored patient data assembled in the database in the server, and to select and prioritize patients and alerts for health care provisioning. In addition to generating alert notifications, the remote device 808, including the server and the interconnected clients, may also execute a follow-up scheme by sending follow-up requests to the one or more ambulatory medical devices, or by sending a message or other communication to the patient 801 (e.g., the patient), clinician or authorized third party as a compliance notification.

The communication network 807 can provide wired or wireless interconnectivity. In an example, the communication network 807 can be based on the Transmission Control Protocol/Internet Protocol (TCP/IP) network communication specification, although other types or combinations of networking implementations are possible. Similarly, other network topologies and arrangements are possible.

One or more of the external device 806 or the remote device 808 can output the detected medical events to a system user, such as the patient or a clinician, or to a process including, for example, an instance of a computer program executable in a microprocessor. In an example, the process can include an automated generation of recommendations for anti-arrhythmic therapy, or a recommendation for further diagnostic test or treatment. In an example, the external device 806 or the remote device 808 can include a respective display unit for displaying the physiologic or functional signals, or alerts, alarms, emergency calls, or other forms of warnings to signal the detection of arrhythmias. In some examples, the external system 805 can include an external data processor configured to analyze the physiologic or functional signals received by the one or more ambulatory medical devices, and to confirm or reject the detection of arrhythmias. Computationally intensive algorithms, such as machine-learning algorithms, can be implemented in the external data processor to process the data retrospectively to detect cardia arrhythmias.

Portions of the one or more ambulatory medical devices or the external system 805 can be implemented using hardware, software, firmware, or combinations thereof. Portions of the one or more ambulatory medical devices or the external system 805 can be implemented using an application-specific circuit that can be constructed or configured to perform one or more functions or can be implemented using a general-purpose circuit that can be programmed or otherwise configured to perform one or more functions. Such a general-purpose circuit can include a microprocessor or a portion thereof, a microcontroller or a portion thereof, or a programmable logic circuit, a memory circuit, a network interface, and various components for interconnecting these components. For example, a “comparator” can include, among other things, an electronic circuit comparator that can be constructed to perform the specific function of a comparison between two signals or the comparator can be implemented as a portion of a general-purpose circuit that can be driven by a code instructing a portion of the general-purpose circuit to perform a comparison between the two signals. “Sensors” can include electronic circuits configured to receive information and provide an electronic output representative of such received information.

The therapy device 810 can be configured to send information to or receive information from one or more of the ambulatory medical devices or the external system 805 using the communication link 811. In an example, the one or more ambulatory medical devices, the external device 806, or the remote device 808 can be configured to control one or more parameters of the therapy device 810. The external system 805 can allow for programming the one or more ambulatory medical devices and can receives information about one or more signals acquired by the one or more ambulatory medical devices, such as can be received via a communication link 811. The external system 805 can include a local external implantable medical device programmer. The external system 805 can include a remote patient management system that can monitor patient status or adjust one or more therapies such as from a remote location.

In certain examples, event storage can be triggered, such as received physiologic information or in response to one or more detected events or determined parameters meeting or exceeding a threshold (e.g., a static threshold, a dynamic threshold, or one or more other thresholds based on patient or population information, etc.). Information sensed or recorded in the high-power mode can be transitioned from short-term storage, such as in a loop recorder, to long-term or non-volatile memory, or in certain examples, prepared for communication to an external device separate from the medical device. In an example, cardiac electrical or cardiac mechanical information leading up to and in certain examples including the detected atrial fibrillation event can be stored, such as to increase the specificity of detection. In an example, multiple loop recorder windows (e.g., 2-minute windows) can be stored sequentially. In systems without early detection, to record this information, a loop recorder with a longer time period would be required at substantial additional cost (e.g., power, processing resources, component cost, amount of memory, etc.). Storing multiple windows using this early detection leading up to a single event can provide full event assessment with power and cost savings, in contrast to the longer loop recorder windows. In addition, the early detection can trigger additional parameter computation or storage, at different resolution or sampling frequency, without unduly taxing finite system resources.

In certain examples, one or more alerts can be provided, such as to the patient, to a clinician, or to one or more other caregivers (e.g., using a patient smart watch, a cellular or smart phone, a computer, etc.), such as in response to the transition to the high-power mode, in response to the detected event or condition, or after updating or transmitting information from a first device to a remote device. In other examples, the medical device itself can provide an audible or tactile alert to warn the patient of the detected condition. For example, the patient can be alerted in response to a detected condition so they can engage in corrective action, such as sitting down, etc.

In certain examples, a therapy can be provided in response to the detected condition. For example, a pacing therapy can be provided, enabled, or adjusted, such as to disrupt or reduce the impact of the detected atrial fibrillation event. In other examples, delivery of one or more drugs (e.g., a vasoconstrictor, pressor drugs, etc.) can be triggered, provided, or adjusted, such as using a drug pump, in response to the detected condition, alone or in combination with a pacing therapy, such as that described above, such as to increase arterial pressure, maintain cardiac output, and to disrupt or reduce the impact of the detected atrial fibrillation event.

In certain examples, physiologic information of a patient can be sensed, such as by one or more sensors located within, on, or proximate to the patient, such as a cardiac sensor, a heart sound sensor, or one or more other sensors described herein. For example, cardiac electrical information of the patient can be sensed using a cardiac sensor. In other examples, cardiac acceleration information of the patient can be sensed using a heart sound sensor. The cardiac sensor and the heart sound sensor can be components of one or more (e.g., the same or different) medical devices (e.g., an implantable medical device, an ambulatory medical device, etc.). Timing metrics between different features (e.g., first and second cardiac features, etc.) can be determined, such as by a processing circuit of the cardiac sensor or one or more other medical devices or medical device components, etc. In certain examples, the timing metric can include an interval or metric between first and second cardiac features of a first cardiac interval of the patient (e.g., a duration of a cardiac cycle or interval, a QRS width, etc.) or between first and second cardiac features of respective successive first and second cardiac intervals of the patient. In an example, the first and second cardiac features include equivalent detected features in successive first and second cardiac intervals, such as successive R waves (e.g., an R-R interval, etc.) or one or more other features of the cardiac electrical signal, etc.

Heart sounds are recurring mechanical signals associated with cardiac vibrations or accelerations from blood flow through the heart or other cardiac movements with each cardiac cycle or interval and can be separated and classified according to activity associated with such vibrations, accelerations, movements, pressure waves, or blood flow. Heart sounds include four major features: the first through the fourth heart sounds (S1 through S4, respectively). The first heart sound (S1) is the vibrational sound made by the heart during closure of the atrioventricular (AV) valves, the mitral valve and the tricuspid valve, and the opening of the aortic valve at the beginning of systole, or ventricular contraction. The second heart sound (S2) is the vibrational sound made by the heart during closure of the aortic and pulmonary valves at the beginning of diastole, or ventricular relaxation. The third and fourth heart sounds (S3, S4) are related to filling pressures of the left ventricle during diastole. An abrupt halt of early diastolic filling can cause the third heart sound (S3). Vibrations due to atrial kick can cause the fourth heart sound (S4). Valve closures and blood movement and pressure changes in the heart can cause accelerations, vibrations, or movement of the cardiac walls that can be detected using an accelerometer or a microphone, providing an output referred to herein as cardiac acceleration information.

In an example, heart sound signal portions, or values of respective heart sound signals for a cardiac interval, can be detected as amplitudes occurring with respect to one or more cardiac electrical features or one or more energy values with respect to a window of the heart sound signal, often determined with respect to one or more cardiac electrical features. For example, the value and timing of an S1 signal can be detected using an amplitude or energy of the heart sound signal occurring at or about the R wave of the cardiac interval. An S4 signal portion can be determined, such as by a processing circuit of the heart sound sensor or one or more other medical devices or medical device components, etc. In certain examples, the S4 signal portion can include a filtered signal from an S4 window of a cardiac interval. In an example, the S4 interval can be determined as a set time period in the cardiac interval with respect to one or more other cardiac electrical or mechanical features, such as forward from one or more of the R wave, the T wave, or one or more features of a heart sound waveform, such as the first, second, or third heart sounds (S1, S2, S3), or backwards from a subsequent R wave or a detected S1 of a subsequent cardiac interval. In certain examples, the length of the S4 window can depend on heart rate or one or more other factors. In an example, the timing metric of the cardiac electrical information can be a timing metric of a first cardiac interval, and the S4 signal portion can be an S4 signal portion of the same first cardiac interval.

In an example, a heart sound parameter can include information of or about multiple of the same heart sound parameter or different combinations of heart sound parameters over one or more cardiac cycles or a specified time period (e.g., 1 minute, 1 hour, 1 day, 1 week, etc.). For example, a heart sound parameter can include a composite S1 parameter representative of a plurality of S1 parameters, for example, over a certain time period (e.g., a number of cardiac cycles, a representative time period, etc.).

In an example, the heart sound parameter can include an ensemble average of a particular heart sound over a heart sound waveform, such as that disclosed in the commonly assigned Siejko et al. U.S. Pat. No. 7,115,096 entitled “THIRD HEART SOUND ACTIVITY INDEX FOR HEART FAILURE MONITORING,” or in the commonly assigned Patangay et al. U.S. Pat. No. 7,853,327 entitled “HEART SOUND TRACKING SYSTEM AND METHOD,” each of which are hereby incorporated by reference in their entireties, including their disclosures of ensemble averaging an acoustic signal and determining a particular heart sound of a heart sound waveform. In other examples, the signal receiver circuit can receive the at least one heart sound parameter or composite parameter, such as from a heart sound sensor or a heart sound sensor circuit.

In an example, cardiac electrical information of the patient can be received, such as using a signal receiver circuit of a medical device, from a cardiac sensor (e.g., one or more electrodes, etc.) or cardiac sensor circuit (e.g., including one or more amplifier or filter circuits, etc.). In an example, the received cardiac electrical information can include the timing metric between the first and second cardiac features of the patient.

In an example, cardiac acceleration information of the patient can be received, such as using the same or different signal receiver circuit of the medical device, from a heart sound sensor (e.g., an accelerometer, etc.) or heart sound sensor circuit (e.g., including one or more amplifier or filter circuits, etc.). In an example, the received cardiac acceleration information can include the S4 signal portion occurring between the first and second cardiac features of the patient. In certain examples, additional physiologic information can be received, such as one or more of heart rate information, activity information of the patient, or posture information of the patient, from one or more other sensor or sensor circuits.

In certain examples, a high-power mode can be in contrast to a low-power mode, and can include one or more of: enabling one or more additional sensors, transitioning from a low-power sensor or set of sensors to a higher-power sensor or set of sensors, triggering additional sensing from one or more additional sensors or medical devices, increasing a sensing frequency or a sensing or storage resolution, increasing an amount of data to be collected, communicated (e.g., from a first medical device to a second medical device, etc.), or stored, triggering storage of currently available information from a loop recorder in long-term storage or increasing the storage capacity or time period of a loop recorder, or otherwise altering device behavior to capture additional or higher-resolution physiologic information or perform more processing, etc.

Additionally, or alternatively, event storage can be triggered. Information sensed or recorded in the high-power mode can be transitioned from short-term storage, such as in a loop recorder, to long-term or non-volatile memory, or in certain examples, prepared for communication to an external device separate from the medical device. In an example, cardiac electrical or cardiac mechanical information leading up to and in certain examples including the detected atrial fibrillation event can be stored, such as to increase the specificity of detection. In an example, multiple loop recorder windows (e.g., 2-minute windows) can be stored sequentially. In systems without early detection, to record this information, a loop recorder with a longer time period would be required at substantial additional cost (e.g., power, processing resources, component cost, etc.).

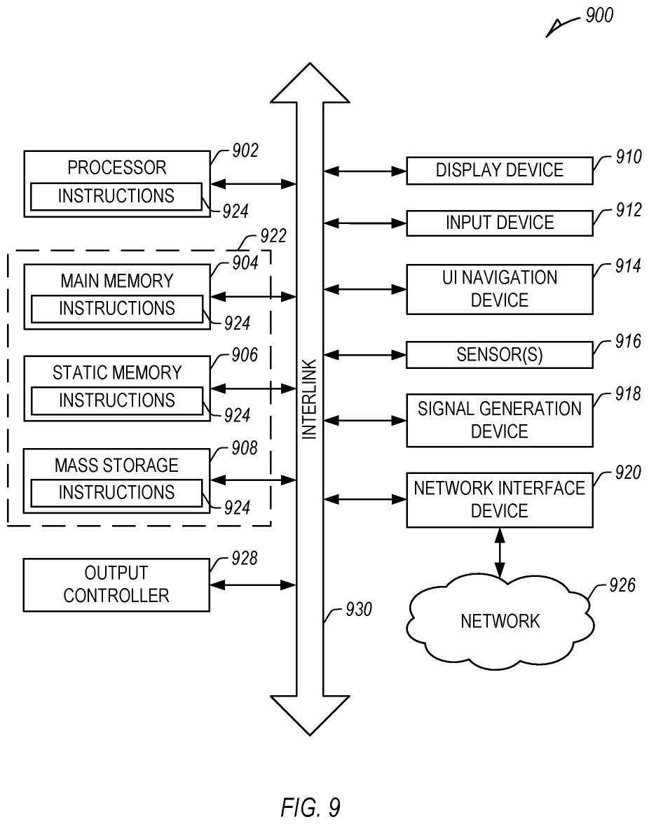

FIG. 9 illustrates a block diagram of an example machine 900 upon which any one or more of the techniques (e.g., methodologies) discussed herein may perform. Portions of this description may apply to the computing framework of one or more of the medical devices described herein, such as the implantable medical device, the external programmer, etc. Further, as described herein with respect to medical device components, systems, or machines, such may require regulatory-compliance not capable by generic computers, components, or machinery.

Examples, as described herein, may include, or may operate by, logic or a number of components, or mechanisms in the machine 900. Circuitry (e.g., processing circuitry, an assessment circuit, etc.) is a collection of circuits implemented in tangible entities of the machine 900 that include hardware (e.g., simple circuits, gates, logic, etc.). Circuitry membership may be flexible over time. Circuitries include members that may, alone or in combination, perform specified operations when operating. In an example, hardware of the circuitry may be immutably designed to carry out a specific operation (e.g., hardwired). In an example, the hardware of the circuitry may include variably connected physical components (e.g., execution units, transistors, simple circuits, etc.) including a machine-readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuitry in hardware via the variable connections to carry out portions of the specific operation when in operation. Accordingly, in an example, the machine-readable medium elements are part of the circuitry or are communicatively coupled to the other components of the circuitry when the device is operating. In an example, any of the physical components may be used in more than one member of more than one circuitry. For example, under operation, execution units may be used in a first circuit of a first circuitry at one point in time and reused by a second circuit in the first circuitry, or by a third circuit in a second circuitry at a different time. Additional examples of these components with respect to the machine 900 follow.

In alternative embodiments, the machine 900 may operate as a standalone device or may be connected (e.g., networked) to other machines. In a networked deployment, the machine 900 may operate in the capacity of a server machine, a client machine, or both in server-client network environments. In an example, the machine 900 may act as a peer machine in peer-to-peer (P2P) (or other distributed) network environment. The machine 900 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

The machine 900 (e.g., computer system) may include a hardware processor 902 (e.g., a central processing unit (CPU), a graphics processing unit (GPU), a hardware processor core, or any combination thereof), a main memory 904, a static memory 906 (e.g., memory or storage for firmware, microcode, a basic-input-output (BIOS), unified extensible firmware interface (UEFI), etc.), and mass storage 908 (e.g., hard drive, tape drive, flash storage, or other block devices) some or all of which may communicate with each other via an interlink 930 (e.g., bus). The machine 900 may further include a display unit 910, an input device 912 (e.g., a keyboard), and a user interface (UI) navigation device 914 (e.g., a mouse). In an example, the display unit 910, input device 912, and UI navigation device 914 may be a touch screen display. The machine 900 may additionally include a signal generation device 918 (e.g., a speaker), a network interface device 920, and one or more sensors 916, such as a global positioning system (GPS) sensor, compass, accelerometer, or one or more other sensors. The machine 900 may include an output controller 928, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

Registers of the hardware processor 902, the main memory 904, the static memory 906, or the mass storage 908 may be, or include, a machine-readable medium 922 on which is stored one or more sets of data structures or instructions 924 (e.g., software) embodying or utilized by any one or more of the techniques or functions described herein. The instructions 924 may also reside, completely or at least partially, within any of registers of the hardware processor 902, the main memory 904, the static memory 906, or the mass storage 908 during execution thereof by the machine 900. In an example, one or any combination of the hardware processor 902, the main memory 904, the static memory 906, or the mass storage 908 may constitute the machine-readable medium 922. While the machine-readable medium 922 is illustrated as a single medium, the term “machine-readable medium” may include a single medium or multiple media (e.g., a centralized or distributed database, and/or associated caches and servers) configured to store the one or more instructions 924.

The term “machine-readable medium” may include any medium that is capable of storing, encoding, or carrying instructions for execution by the machine 900 and that cause the machine 900 to perform any one or more of the techniques of the present disclosure, or that is capable of storing, encoding, or carrying data structures used by or associated with such instructions. Non-limiting machine-readable medium examples may include solid-state memories, optical media, magnetic media, and signals (e.g., radio frequency signals, other photon-based signals, sound signals, etc.). In an example, a non-transitory machine-readable medium comprises a machine-readable medium with a plurality of particles having invariant (e.g., rest) mass, and thus are compositions of matter. Accordingly, non-transitory machine-readable media are machine-readable media that do not include transitory propagating signals. Specific examples of non-transitory machine-readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

The instructions 924 may be further transmitted or received over a communications network 926 using a transmission medium via the network interface device 920 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax®), IEEE 802.15.4 family of standards, peer-to-peer (P2P) networks, among others. In an example, the network interface device 920 may include one or more physical jacks (e.g., Ethernet, coaxial, or phone jacks) or one or more antennas to connect to the communications network 926. In an example, the network interface device 920 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding, or carrying instructions for execution by the machine 900, and includes digital or analog communications signals or other intangible medium to facilitate communication of such software. A transmission medium is a machine-readable medium.

Various embodiments are illustrated in the figures above. One or more features from one or more of these embodiments may be combined to form other embodiments. Method examples described herein can be machine or computer-implemented at least in part. Some examples may include a computer-readable medium or machine-readable medium encoded with instructions operable to configure an electronic device or system to perform methods as described in the above examples. An implementation of such methods can include code, such as microcode, assembly language code, a higher-level language code, or the like. Such code can include computer readable instructions for performing various methods. The code can form portions of computer program products. Further, the code can be tangibly stored on one or more volatile or non-volatile computer-readable media during execution or at other times.

The above detailed description is intended to be illustrative, and not restrictive. The scope of the disclosure should, therefore, be determined with references to the appended claims, along with the full scope of equivalents to which such claims are entitled.

What is claimed is:

1. A medical device system, comprising:
a signal receiver circuit configured to receive physiologic information of a patient;
an assessment circuit configured to adjust one or more determined boundaries of a display of the received physiologic information based on different first and second ranges of physiologic information, including to:
determine first and second boundaries of a first range of the received physiologic information; and
determine first and second boundaries of a second range of the received physiologic information, the second range within the first range; and
an output circuit configured to provide the one or more determined boundaries of the display for presentation to a user, the display comprising the determined first and second boundaries of the first range and the determined first and second boundaries of the second range.

2. The medical device system of claim 1, wherein the first boundary of the first range comprises a target value of the received physiologic information,
wherein the assessment circuit is configured to determine the target value as a function of a clinical target value and the received physiologic information.

3. The medical device system of claim 2, wherein the assessment circuit is configured to update the one or more boundaries at a regular interval using additionally received physiologic information,
wherein to update the one or more boundaries comprises to update the target value in a direction towards the clinical target, wherein the assessment circuit is restricted from updating the target value in a direction away from the clinical target.

4. The medical device system of claim 2, wherein the assessment circuit is configured to determine the target value as a function of the clinical target value and a first extremum value of the received physiologic information occurring over the first range.

5. The medical device system of claim 2, wherein the second boundary of the first range comprises a second extremum value of the received physiologic information occurring over the first range, wherein one of the first and second extremum value is a maximum value and another is a minimum value,
wherein the first and second boundaries of the second range comprise respective minimum and maximum values of the received physiologic information occurring over the second range.

6. The medical device system of claim 5, wherein the output is configured to provide the first boundary as an arrow indicating a desired direction and the target value of the received physiologic information in contrast to the second range of the received physiologic information.

7. The medical device system of claim 1, wherein the first and second boundaries of the first range comprise respective minimum and maximum values of the received physiologic information occurring over the first range,
wherein the first and second boundaries of the second range comprise respective minimum and maximum values of the received physiologic information occurring over the second range.

8. The medical device system of claim 1, wherein the display comprises the determined first and second boundaries of the first range, the determined first and second boundaries of the second range, and a single representative current value of the received physiologic information.

9. The medical device system of claim 8, wherein the single representative current value of the received physiologic information comprises a most recent daily value of the received physiologic information.

10. The medical device system of claim 8, wherein the first range comprises a first number of daily values,
wherein the second range comprises a second number of daily values, the second number of daily values being more than two days and less than three weeks,
wherein the first number of daily values is greater than the second number of daily values.

11. The medical device system of claim 10, wherein the received physiologic information comprises daily values of the received physiologic information,
wherein the single representative current value of the received physiologic information comprises a most recent daily value of the received physiologic information,
wherein the first range comprises a previous six months of daily values with respect to the most recent daily value,
wherein the second range comprises a previous two weeks of daily values with respect to the most recent daily value.

12. The medical device system of claim 1, wherein the physiologic information includes at least one of respiration information of the patient, cardiac electrical information of the patient, impedance information of the patient, cardiac acceleration information of the patient, or sleep incline information of the patient.

13. The medical device system of claim 12, wherein the physiologic information includes a plurality of daily respiration values,
wherein the display comprises the determined first and second boundaries of the first range, the determined first and second boundaries of the second range, and a most recent daily respiration value.

14. A method, comprising:
receiving, using a signal receiver circuit, physiologic information of a patient;
adjusting, using an assessment circuit, one or more determined boundaries of a display of the received physiologic information based on different first and second ranges of physiologic information, the adjusting comprising:
determining first and second boundaries of a first range of the received physiologic information; and
determining first and second boundaries of a second range of the received physiologic information, the second range within the first range; and
providing, using an output circuit, the one or more determined boundaries of the display for presentation to a user, the display comprising the determined first and second boundaries of the first range and the determined first and second boundaries of the second range.

15. The method of claim 14, wherein determining the first boundary of the first range comprises determining a target value of the received physiologic information as a function of a clinical target value and the received physiologic information.

16. The method of claim 15, comprising updating, using the assessment circuit, the one or more boundaries at a regular interval using additionally received physiologic information, including updating the target value in a direction towards the clinical target and restricting updating the target value in a direction away from the clinical target.

17. The method of claim 15, wherein determining the target value includes as a function of the clinical target value and a first extremum value of the received physiologic information occurring over the first range.

18. The method of claim 15, wherein determining the second boundary of the first range comprises determining a second extremum value of the received physiologic information occurring over the first range, wherein one of the first and second extremum value is a maximum value and another is a minimum value,
wherein determining the first and second boundaries of the second range comprise determining respective minimum and maximum values of the received physiologic information occurring over the second range.

19. The method of claim 18, wherein providing the display comprises providing the first boundary as an arrow indicating a desired direction, and the target value, of the received physiologic information, in contrast to the second range of the received physiologic information.

20. The method of claim 14, wherein determining the first and second boundaries of the first range comprise determining respective minimum and maximum values of the received physiologic information occurring over the first range,
wherein determining the first and second boundaries of the second range comprise determining respective minimum and maximum values of the received physiologic information occurring over the second range,
wherein the display comprises the determined first and second boundaries of the first range, the determined first and second boundaries of the second range, and a single representative current value of the received physiologic information.
